(19) United States
(12) Patent Application Publication
(10) Pub. No.: US 2009/0149802 A1
(43) Pub. Date: Jun. 11, 2009

(54) Surgical System Including a Trap for Noise-Inducing Materials

(76) Inventor: Ross Peter Jones, Cambridge (GB)

Correspondence Address: Bausch & Lomb Incorporated, One Bausch & Lomb Place, Rochester, NY, 14604-2701, US

(21) Appl. No.: 11/952,183

(22) Filed: Dec. 7, 2007

(51) Int. Cl. A61F 9/007; A61M 1/00

(52) U.S. Cl. 604/35

(57) ABSTRACT

The present invention provides a surgical system 10 a source of irrigation fluid 20, a collection cassette 30, a handpiece 50 applied to a surgical area for infusing irrigation fluid and for aspirating a biological material, first and second conduits (60 and 62) connecting the handpiece 50 to each of the source of irrigation fluid 20 and the collection cassette 30, a monitoring device 70 and means for trapping materials 80 that cause signal disruption to the monitoring device 70.

### FIELD

The present invention relates generally to a system useful for various surgical procedures. More specifically, it relates to a surgical system having means for trapping materials causing noise to a flow monitoring device in an ophthalmic surgical procedure.

### BACKGROUND

A cataract is an opacity that develops in the crystalline lens of the eye or in its envelope. One medical procedure to remove a cataract-affected lens is phacoemulsification (phaco) using ultrasonic sound to break up or emulsify the cataract. A phacoemulsification machine typically includes a handpiece with both irrigation and aspiration functions. A phaco handpiece aspirates in emulsified fluids and simultaneously replaces those aspirated fluids with balanced salt solution (BSS) to maintain a proper pressure of the antenor chamber of the patient's eye. Such a handpiece is connected to a pump generating negative pressure or vacuum to drive aspiration, by which debris from the eye flow through a tube to means for collection such as a cassette, a bag and a bottle.

A common and dangerous occurrence in ophthalmic surgery is “post-occlusion surge.” During ophthalmic surgery, particularly cataract surgery, as the lens is broken-up and emulsified, such as during phacoemulsification, irrigation fluid is constantly infused into the surgical site and the fluid and emulsified tissue are aspirated away from the surgical site through the phaco handpiece. On occasion bits of tissue are larger than the aspiration lumen in the phaco handpiece, which can result in a clogged aspiration conduit. As long as the aspiration conduit remains clogged, a negative pressure builds up throughout the aspiration system. Then, after the clog has been removed, the system can experience what is commonly referred to as surge. Post-occlusion surge can cause serious damage to a patient's eye, such as by rupturing a capsular bag and allowing vitreous to leak from the eye's posterior into the eye's anterior chamber or cause irreparable damage to the cornea's endothelial cells. Generally speaking, endothelial cells are not regenerated naturally and it is crucial to prevent post-occlusion surge in an ophthalmic operation.

One attempt to prevent post-occlusion surge is to provide an automated surgical system monitoring one or more parameters at predetermined sites and controlling operation of the surgical system in accordance with the collected information. For example, U.S. Pat. No. 5,733,256 mentions a surgical system for phacoemulsification comprising a surgical sensing module to monitor fluid flow parameters which is placed in a close proximity of less than 8 inches with the surgical handpiece. The invention aims to improve the speed and precision of the measurement and control of fluid flow and pressure parameters.

Monitoring parameters within a surgical system is also important for other purposes such as, for example, control of an irrigation flow rate. U.S. Pat. No. 5,810,765 describes an irrigation/aspiration apparatus comprising irrigation flow rate control means for supplying a flow rate signal and an aspiration signal. The apparatus is designed to suppress the variation of the pressure in the anterior chamber of the patient's eye during the cataract operation.

However, the above-mentioned inventions failed to address that it is difficult in practice to monitor physical or chemical parameters accurately from an aspiration surgical system because the aspirated surgical fluids contain various noise-inducing materials. For example, noise-inducers present in the aspiration conduit include bubbles and viscoelastics. Bubbles are created at the tip of the handpiece during breaking up undesirable biological materials and eventually come into contact with the electrodes of a monitoring device, making the electric circuit open and leading to distortion of the signal. Viscoelastic, which is originated from surgical materials or the patient's eye, contains a polysaccharide carrying surface charges and a contact of viscoelastic with the electrodes causes noise in producing electronic signals. The noisy signals are not desirable to a surgical system, particularly an automated surgical system, because the controller or main console cannot properly interpret the signal therefrom. As a result, the controller may send a wrong direction leading to unintended operation or failure to prevent post-occlusion surge.

### SUMMARY OF THE INVENTION

It is therefore one of the objects of this invention to provide a surgical system capable of monitoring various parameters accurately by removing materials causing noise to a monitoring device.

In one embodiment, it is provided a surgical system comprising a source of irrigation fluid, a collection cassette, a handpiece applied to a surgical area for infusing irrigation fluid and for aspirating a biological material, a conduit connecting the handpiece to each of the source of irrigation fluid and the collection cassette, a monitoring device and means for trapping materials that cause signal disruption to the monitoring device.

In another embodiment, it is provided a surgical system for ophthalmic surgery comprising a source of irrigation fluid, a collection cassette, a handpiece applied to a surgical area for infusing irrigation fluid and for aspirating a biological material, a conduit connecting the handpiece to each of the source of irrigation fluid and the collection cassette, a monitoring device and means for trapping materials that cause signal disruption to the monitoring device.

Yet in another embodiment, it is provided an ophthalmic surgical system for cataract surgery comprising a source of irrigation fluid, a collection cassette, a handpiece applied to a surgical area for infusing irrigation fluid and for aspirating a biological material, a conduit connecting the handpiece to each of the source of irrigation fluid and the collection cassette, a monitoring device and means for trapping materials that cause signal disruption to the monitoring device.

### BRIEF DESCRIPTION OF DRAWINGS

FIG. 1 is a diagrammatic view of one embodiment of a surgical system, in accordance with the present invention;

FIG. 2 is a diagrammatic view of a trap, in accordance with the present invention; and

FIG. 3 is a cross-section of an embodiment of a trap in accordance with the present invention.

### DETAILED DESCRIPTION

The following description is merely exemplary in nature and is not intended to limit the present disclosure, application, or uses.

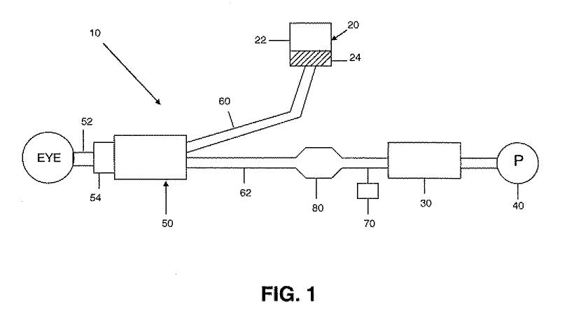

Referring to FIG. 1, the surgical system 10 comprises a source of irrigation fluid 20, a collection cassette 30, a vacuum pump 40, a surgical handpiece 50, conduits 60 and 62 connecting the surgical handpiece to each of the irrigation fluid source and the vacuum pump/the collection cassette, a monitoring device 70 and trap 80 for filtering materials that cause signal disruption to the monitoring device 70. The surgical system 10 is particularly useful in ophthalmic surgery where it is necessary to break up and remove undesirable biological materials from the patient's eye. Specifically, the surgical system 10 can be used to remove cataract without causing irreparable damage to the eye.

The source of irrigation fluid 20 typically includes a fluid container 22 and surgical fluid 24. The surgical fluid can be any known surgical fluid and an ordinary skilled person in the art can select proper surgical fluid in accordance with the nature of the surgery to be operated. In an ophthalmic surgical system, the surgical fluid 24 is ophthalmic surgical fluid such as, for example, BSS. Each end of the conduit 60 is connected to the container 20 and the phaco handpiece 50 respectively so that the ophthalmic surgical fluid is delivered to the patient's eye through the irrigation sleeve 54 of the phaco handpiece 50.

The collection cassette 30 typically has a collection chamber and an inlet and an outlet for connection to each of the handpiece 50 and the vacuum pump 40. The collection chamber accommodates biological debris aspirated from the surgical site via the needle means 52 of the handpiece 50 and the aspiration conduit 62. The collection cassette 30 can be selected from any collection means for a surgical system known in the art, regardless of its reusability. Thus, the cassette 30 can be any known reusable or disposable collection means. For safety and sanitary reasons, it may be preferable to select a collection cassette equipped with a fluid level detection device which is designed to prevent overflowing and leaking surgical fluids. The collection cassette 30 is installed in operative association with the handpiece 50 and the pump 40 by any means known in the art.

The vacuum pump 40 is connected to the collection cassette 30 and the handpiece 40 through the aspiration conduit 62 to provide the aspiration system comprising the handpiece, the conduit and the collection cassette with negative pressure or vacuum. The vacuum pump 40 can be any pump known in the art as long as it is suitable for a surgical system including the present surgical system. Preferably, the vacuum pump 40 is one suitable for an ophthalmic surgical system. Examples of a pump applicable to the present invention are, but not limited to, a venturi pump, a rotary vane pump, a diaphragm pump, a liquid ring pump, a piston pump, a scroll pump, a screw pump, an Wankel pump, an external vane pump, a booster pump, a multistage roots pump, a peristaltic pump and a Toepler pump.

The surgical handpiece 50 can be a conventional phacoemulsification handpiece comprising an operative tip, a needle 52 and an annular sleeve for irrigation 54 surrounding the needle. The surgical handpiece 50 is placed on or into the surgical site to remove undesirable biological materials. In an ophthalmic surgical system, for example, the phaco handpiece 50 is inserted though an incision in an eye, and the operative tip coupled to an energy source applies energy, such as ultra-sound and laser, to the surgical site to break up undesirable biological materials such as cataract. The surgical fluid 24 is infused into the surgical site through the annular sleeve 54 and the needle 52 simultaneously aspirates fluids containing the undesirable materials away from the eye.

The surgical system 10 typically requires two separate conduits 60 and 62 for the irrigation and aspiration system. The irrigation conduit 60 connects the surgical handpiece 50 to the irrigation fluid source 20 to provide the surgical site with the surgical fluid 24 such as BSS. The irrigation system may contain one or more valves placeable between the handpiece 50 and the irrigation fluid source 20 to control the irrigation flow rate, thereby helping maintenance of a proper pressure of the surgical site.

The aspiration conduit 62 connects, for example, the surgical handpiece 50 to the collection cassette 30 and then to the vacuum pump 40, but it is obvious to an ordinary skilled person in the art that it is possible to modify the placement and the connection of the aspiration components. The vacuum pump 40 is operatively connected to the collection cassette 30 through the aspiration conduit 62 such that undesirable biological materials from the surgical site are aspirated to the collection cassette 30 for temporary storage and later disposal.

The monitoring device 70 measures physical or chemical parameters, such as pressure, liquid flow rate and gas flow rate, within the surgical system to generate a signal or information necessary to control the system properly. The monitoring device 70 can be any known monitoring device in the art but it may be preferable for the present invention to employ those which have been used in an ophthalmic surgical system. More preferably, the monitoring device 70 is an electromagnetic flow meter, also known as magflow meter, containing means for applying a magnetic field to the monitoring site for a measurement. The monitoring device 70 is attached to a pre-determined point of the aspiration conduit 62 such that the sensing member of the monitoring device is exposed to the aspiration liquids. In one embodiment, the monitoring device 70 is optionally linked to a control device 72 which collects information from the monitoring device and transmits electronic/mechanic signals to pre-determined sites of the surgical system. The surgical system may be computerized with electronic means where the computerized control device analyzes collected parameters and produces a programmed signal in accordance with the calculation.

The trap 80 for filtering materials causing signal disruption to the monitoring device 70 is placed between the surgical handpiece 50 and the monitoring device 70 to remove undesirable materials prior to their contact with the monitoring device 70. The trap 80 may be tailored to capture any specific material coming from the surgical site as required. Regardless of the design, however, the selectivity to a noise-inducing material should be maintained to achieve the goal of the invention. The noise-inducing material may vary depending on the type of the monitoring device 70 employed and the nature of the surgery to be operated. In one embodiment, the monitoring device 70 is a magflow meter for an ophthalmic surgical system and the noise-inducing material is a viscoelastic. The viscoelastic may be originated from either surgical materials such as, for example, intraocular lens, instruments, or the patient's eye such as, for example, vitreous. Therefore, in an embodiment, the means for trapping noise-inducing materials is designed to capture such a viscoelastic.

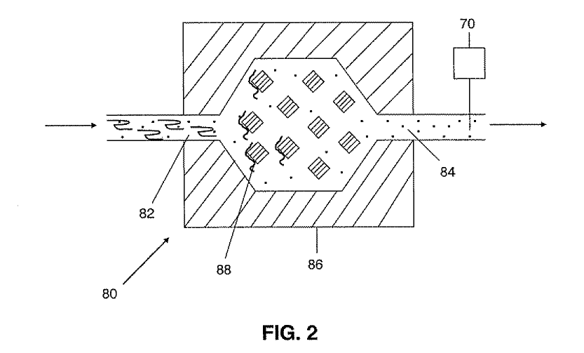

FIG. 2 is an embodiment of trap 80 for filtering materials causing signal disruption where the trap has an inlet 82 connected to the handpiece 50, an outlet 84 to the cassette 30 through the aspiration conduit 62 and a plurality of trapping members 88. The trap 80 is encapsulated with a housing 86 having internal space for trapping in which the trapping members 88 are arrayed to capture noise-inducing materials. The surgical fluids from the handpiece 50 are introduced into the inlet 82 and discharged through the outlet 84 after filtration by the trapping members 88. The internal space of the housing 88 forms a chamber whose width is bigger than those of the inlet and outlet so as to provide enough trapping/flowing space. The trapping members 88 can be arrayed in a number of different patterns so long as the variation does not cause total loss of their trapping capability. In a preferred embodiment, the trapping members 88 are placed in two or more rows along the aspiration flow direction to enable layered capture of the materials and each row has two or more trapping members to make a plurality of gaps between the members for fluid passage. The number of the trap 88 may be proportional to their trapping capacity to a certain degree and thus the number can be determined based on a predicted trapping volume. It is preferable to prepare one unit of trap that can last at least one entire operation without replacement. The sizes of the trapping member and the gap can be determined by an ordinary skilled person in the art based on the materials to be captured and the flow rate. In an embodiment, the trap 80 optionally includes an indicator showing its remaining trapping capacity so that the operator or assistant can determine a replacing timing of the trap.

The present invention is particularly useful in trapping or capturing noise-inducing, i.e., signal disruption materials that are distinguished from the carrier fluid by their viscosity, cohesion or surface affinity to the material of trapping member 88. The trap 80 requires a pressure to push the signal disruption material through gaps between trapping members 88 that is much greater than a pressure required to push the carrier fluid through the gaps. The carrier fluid is typically balanced salt solution and the signal disruption material includes viscoelastic or other materials and tissue that may interfere with the monitoring device 70.

It is important that the trap allow the pressure of the flow to push the signal disruption material through the gaps, so that fluid does not build up excessively up stream of the trap 80. For example, when a row of trap 80 is completely filled with signal disruption material, the trap must allow the pressure to clear a path to the next row. It is noted that the material shown in FIG. 2 is not only trapped on the members 88, but mainly will become trapped between members 88.

The present invention can be distinguished from prior art particle traps where particles are trapped because they are physically too large to fit through a gap. In a particle trap, once a particle is trapped it cannot move further down stream, The present inventive trap allows trapped material to move down stream to the next row once a previous row has become filled. This allows the present inventive trap to provide a large trapping capacity for a relatively small cross-sectional area of the trap. Unlike prior art particle traps that require a large cross-sectional (e.g. filter paper) area or large volume (e.g. porous foam filter or packed bed filter); the present invention does not need to be deep in the direction parallel to the members 88. The capacity of the present inventive trap can be increased by making it longer (adding rows of members 88) without increasing its cross-sectional area.

Another advantage of the present invention over prior art particle traps, is that the present invention will not become clogged and prevent the flow of fluid like particle traps will. Once the trap 80 fills up, fluid, including signal disruption material, will continue to flow through traps 80.

In an embodiment, the viscosity and cohesion of signal-disrupting materials 90 such as viscoelastic causes automatic trapping at the trapping members 88. Due to the pressure difference and the narrow opening of the needle 52, viscoelastic materials tend to be a string-like structure upon passage through the needle. When a string of the viscoelastic 90 enters the gap between two trapping members 88, it gets stuck, leaving the filtered surgical fluids free to flow through any unblocked paths. Aspiration flow of the filtered fluid then continues through the parallel gaps. When these are all full of the noise-inducing materials, the aspiration flow pushes the materials out of one or two weak gaps. This pushed out materials then gets trapped in the next row of trapping members. Continuing in this manner, the rows of gaps are largely filled in order by the noise-inducing materials, with each row having at least one gap free for the surgical fluids to pass through.

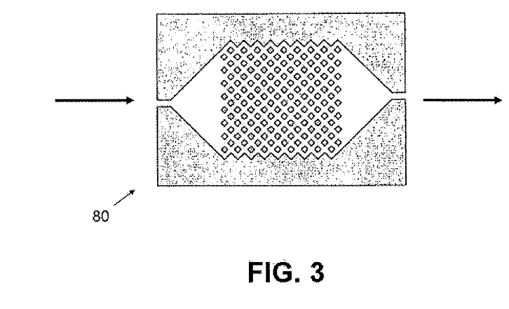

FIG. 3 is a preferred embodiment of means for trapping materials causing signal disruption. The chamber accommodates a plurality of trapping members 88 which are aligned in multiple rows along the aspiration flow direction. The trapping members 88 are lined in an alternating pattern or zigzag pattern such that a material coming out of a gap between two trapping members faces a trapping member in the next row. The pattern may increase the chance for the materials to contact a trapping member, resulting in enhanced trapping efficiency.

The embodiments are described in order to best explain the principles of the invention and its practical application to thereby enable others skilled in the art to best utilize the invention in various embodiments and with various modifications.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims and their equivalents.

What is claimed is:

1. A surgical system comprising
a source of irrigation fluid;
a collection cassette;
a handpiece applied to a surgical area for infusing irrigation fluid and for aspirating a biological material;
first and second conduits connecting the handpiece to each of the source of irrigation fluid and the collection cassette;
a monitoring device; and
a trap, including a housing with an inlet connected to the handpiece and an outlet connected to the cassette through the conduit and a plurality of trapping members placed inside the housing to capture the materials that cause signal disruption to the monitoring device, wherein a pressure required to push the signal disruption materials through gaps between the trapping members, is much greater than a pressure required to push a carrier fluid, carrying the signal disruption materials, through the gaps.

2. The surgical system according to claim 1, wherein the trapping members are arrayed in two or more rows along an aspiration flow direction and each of the rows has at least two trapping members.

3. The surgical system according to claim 2, wherein the trapping members are lined in an alternating pattern such that a material coming out of a gap between two trapping members faces a trapping member in the next row.

4. The surgical system according claim 1, wherein the surgical system is for ophthalmic surgery and the handpiece is a phacoemulsification handpiece applied to a patient's eye.

5. The surgical system according claim 1, wherein the material to be trapped is viscoelastic.

6. A trap for capturing signal disruption materials contained within a carrier fluid comprising:
a housing having an inlet for connection to a surgical handpiece and an outlet for connection to a collection cassette; and
a plurality of trapping members placed inside the housing to capture the signal disruption materials that cause signal disruption to a monitoring device, wherein a pressure required to push the signal disruption materials through gaps between the trapping members is greater than a pressure required to push the carrier fluid through the gaps.

7. The trap of claim 6, wherein the trapping members are arrayed in two or more rows along an aspiration flow direction and each of the rows has at least two trapping members.

8. The trap of claim 7, wherein the trapping members are lined in an alternating pattern such that a material coming out of a gap between two trapping members faces a trapping member in the next row.

9. The trap of claim 6, wherein the material to be trapped is viscoelastic.
